(19) United States
(12) Patent Application Publication
(10) Pub. No.: US 2026/0218115 A1
(43) Pub. Date: Jul. 30, 2026

(54) BIOFILMS ENCAPSULATED WITH SOL-GEL

(71) Applicant: University of Iowa Research Foundation, Iowa City, IA (US)

(72) Inventors: Timothy E. Mattes, Iowa City, IA (US); Qin Dong, Iowa City, IA (US)

(21) Appl. No.: 19/462,794

(22) Filed: Jan. 28, 2026

Related U.S. Application Data
(60) Provisional application No. 63/750,625, filed on Jan. 28, 2025.

(51) Int. Cl. C12N 1/20; B01D 53/70; B01D 53/85; B09C 1/10; C02F 3/10; C02F 3/34; C02F 101/36

(52) U.S. Cl. CPC C12N 1/20; B01D 53/70; B01D 53/85; B09C 1/10; C02F 3/106; C02F 3/348; B01D 2251/95; C02F 2101/363

(57) ABSTRACT

A method and composition for enhancing the stability and biodegradation potential of biofilms used in environmental remediation. The described approach involves encapsulating biofilm-enriched black carbon (BC) with a sol-gel matrix formed from tetraethyl orthosilicate (TEOS) and methyltriethoxysilane (MTES), optionally including additives such as glycerol, polyethylene glycol (PEG400), and polyvinylpyrrolidone (PVP). The sol-gel encapsulation provides a protective, porous, and mechanically stable coating that prevents biofilm washout and maintains cell viability under adverse environmental conditions, such as high salinity and continuous shear force. The encapsulated biofilms demonstrate prolonged degradation of pollutants, including polychlorinated biphenyls (PCBs), over extended periods without additional carbon sources. The sol-gel matrix allows efficient mass transfer of contaminants to the biofilms while preserving biofilm integrity. This technology is applicable to in situ sediment remediation, bioreactors, and other bioremediation applications, offering a robust solution for mitigating environmental pollutants and enhancing public health and ecosystem safety.

### PRIORITY

This application claims the benefit of the filing date of U.S. provisional application Ser. No. 63/750,625, filed on Jan. 28, 2025, the disclosure of which is incorporated by reference herein.

### STATEMENT REGARDING FEDERALLY FUNDED RESEARCH

This invention was made with government support under grant number P42ES013661 awarded by the National Institutes of Health. The government has certain rights in this invention.

### FIELD OF THE INVENTION

The present disclosure pertains to environmental remediation technologies, specifically focusing on the stabilization and improvement of biofilm-based bioremediation systems through sol-gel encapsulation.

### BACKGROUND

Polychlorinated biphenyls (PCBs) have accumulated and persisted to high levels in sediments decades after being banned. Sediment PCBs can bioaccumulate through food webs and/or expose nearby communities through inhalation (1-5)—an increasingly recognized exposure route (6, 7)—which causes adverse human health effects. (8-10) Lower chlorinated PCBs (LC-PCBs) are more volatile PCBs and can transport from sediment into overlaying water and air; (7) indeed, dredging does not degrade PCBs and can mobilize sediment LC-PCBs. (11-13) Anaerobic reductive dechlorination of higher chlorinated PCBs can also contribute to continuous LC-PCBs emissions from contaminated sediment. (14) LC-PCB emissions can be mitigated through bioaugmentation of aerobic microorganisms. (15) Biofilms grown on black carbons (BCs) are effective in sustaining aerobic PCB-degraders' viability and inducing PCB biodegradation activity, while in contrast suspended cells are less active. (16) Nevertheless, the biomass abundance of bioaugmented PCB-degraders with BCs decreases over time following deployment for in-situ PCB sediment remediation. (14, 17, 18) Unfavorable environmental conditions, including shear force (e.g., external water force) and salinity, can drive biomass washout and create intense stress that threatens cell survivability and activity. (19)

### SUMMARY

Biofilms, such as those made by Paraburkholderia xenovorans LB400, hold the potential to degrade toxins in the environment, such as PCBs in contaminated sediment. Nevertheless, unfavorable environmental conditions (e.g., salinity, temperature, shear force) can interfere with biofilm stability and affect biodegradation potential. Sol-gel encapsulation has been used to protect planktonic cell function due to high material stability and absence of cell washout, but not employed for biofilm protection. Provided herein is a sol-gel application with biofilm-enriched black carbons for prolonged biodegradation. Multiple sol-gel recipes were systematically tuned to coat biofilms and the impact of sol-gel coating on cell survival and pollutant degradation was measured. The developed sol-gel completely encapsulated biofilm-enriched black carbons and produced both high porosity and appropriate pore size that allow pollutant transfer from the surrounding environment to the biofilms. The sol-gel maintained physical integrity under saline conditions (simulating marine/estuary sediments) and continuously applied shear force. Additionally, the encapsulated biofilms degraded benzoate, a proof-of-concept organic molecule, and extended biofilm attachment and cell viability for over three months without a carbon and energy source. This demonstrates that sol-gel helps sustain PCB-degrading biofilms under environmentally relevant conditions. This novel sol-gel application can improve bioaugmentation effectiveness and enhance degradation of environmental pollutants.

In one embodiment, a microbial composition comprises bacteria together with a feedstock, the entire composition being coated with a sol gel. The bacteria may be selected from genera such as Bacillus, Rhodococcus, Arthrobacter, Cycloclasticus, Paraburkholderia or Alcanivorax—including Paraburkholderia xenovorans LB400 or Alcanivorax borkumensis I—and may form one or more biofilms. The feedstock may be black carbon (for example biochar or corn kernel biochar). In some embodiment, the sol gel comprises tetraethyl orthosilicate (TEOS) and methyltriethoxysilane (MTES) in a 0.5:0.5:20 ratio with water, 10 mg of polyvinylpyrrolidone, and 20% glycerol or PEG400 (v/v); the sol gel forms pores larger than contaminant molecules and smaller than bacterial cells and may be applied in one or two coats. The coated composition exhibits enhanced stability compared to an uncoated composition and, in specific embodiments, degrades polychlorinated biphenyls such as PCB4, PCB18 or PCB52.

In another embodiment, a bioremediation method comprises contacting a contaminated environmental sample with the above-described microbial composition. The sample may be soil, water or air, and treatment can be performed in situ.

In a further embodiment, a stabilization method comprises coating a microbial composition—optionally including bacterial biofilms, selected bacteria (e.g., Paraburkholderia xenovorans LB400) and a feedstock (e.g., biochar)—with a sol gel formed from, for example, MTES, TEOS, water, polyvinylpyrrolidone and glycerol or PEG400, optionally applied in two successive coats. This coating method increases composition stability and promotes elevated expression levels of degradation enzymes such as bphA relative to unencapsulated biofilms.

### DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1. Summary of encapsulation procedures to control sol-gel completeness, hardness, and pore structure.

FIGS. 2A1-2D4. SEM imaging of sol-gel encapsulated-BC properties: completeness, observational hardness test, and porosity. No encapsulation: a1, a2, a3, a4; glycerol addition: b1, b2, c1, c2, d1, d2; PEG400 addition: b3, b4, c3, c4, d3, d4. Row “1” and “2” represented the samples made by the same recipe but visualized in different magnifications, and row “3” and “4” also represented the same sample in different magnifications. Note the different magnifications in each image (see scale bar).

FIGS. 3A-3F. SEM images of sol-gel coated black carbons. Subpanels visually portray the effects of each factor on developing a complete, porous, and high observational hardness sol-gel encapsulation. All images were black carbon covered by sol-gel made by different recipes. Recipe-A represents the factor of hydrolysis ratio: TEOS/water=1:20, double sinking; recipe-B represents the factor of MTES addition: TEOS/water=1:20, MTES: 10% of TEOS (v/v), single sinking; recipe-C represents the factor of PVP addition: TEOS/water=1:20, PVP=100 mg, glycerol: 20% of sol solution (v/v), single coating; recipe-D represents the factor of PVP addition: MTES/TEOS/water=0.5:0.5:20, PVP=10 mg, glycerol: 20% of sol solution (v/v), single coating; recipe-E represents the factor of glycerol addition: MTES/TEOS/water=0.5:0.5:20, PVP=10 mg, glycerol: 20% (v/v), two-time coating (tea ball); recipe-F represents the factor of PEG400 addition: MTES/TEOS/water=0.5:0.5:20, PVP=10 mg, PEG400: 20% (v/v), two-time coating (tea ball). Recipe-E and recipe-F were the optimal sol-gel encapsulation recipes=MTES/TEOS/water=0.5:0.5:20, PVP=10 mg, glycerol/PEG400: 20% (v/v), two-time coating (tea ball).

FIGS. 4A-4C. Methyl orange concentration was detected at OD464 through time with corresponding surface pore size. (a) PEG1 represents 6% (v/v) glycerol addition; PEG2 represents 10% (v/v) PEG400 addition; PEG3 represents 20% (v/v) PEG400 addition; PEG4 represents no PEG400 or glycerol addition. (b) MTES 1: 20% (v/v) glycerol; MTES 2: 10% (v/v) PEG400; MTES 3: 20% (v/v) PEG400; MTES 4: no glycerol or PEG400. Gel in (a) was made with only TEOS (TEOS:water=1:21) while gel in (b) had MTES addition (MTES:TEOS:water-1:1.55:11.41). (c) SEM images of sol-gel surface made by PEG 1-4 corresponding to (a).

FIGS. 5A-5D. Cell viability of sol-gel encapsulated biofilm-BC. a) unencapsulated biofilm-BC after three-round of reuse (10 mM benzoate in each round; biofilms were transferred to fresh media for reuse) within 25 days; (b) sol-gel coated biofilms by recipe-A before any treatment; c) sol-gel coated biofilms by recipe-A after three-round of reuse (10 mM benzoate in each round; biofilms were transferred to fresh media for reuse) within 25 days; d) sol-gel coated biofilms by recipe-E after adding only one benzoate spike (10 mM) within three months. Recipe-A: 1:20 of TEOS:water, double sinking, 1:3 sol to phosphate buffer, aging under vacuum; recipe-E: glycerol (20%, v/v), MTES/TEOS/water=0.5:0.5:20, PVP=10 mg, double coating (tea ball) (MTDP-G). Green dots represent live LB400 cells while red dots represent dead LB400 cells. Green continuous pieces in (b) and (d) result from sol-gel sorbing SYTO9 dye and emitting fluorescence.

FIGS. 6A-6C. Benzoate biodegradation performance: (a) comparing benzoate degradation performance between encapsulated biofilm and nonencapsulated biofilm, where biofilm was coated by recipe-A; error bars represent the standard error of biological duplicates; (b) comparing benzoate degradation between recipes with MTES addition and TEOS only, where biofilm was coated by gel plate; (c) comparing benzoate degradation between encapsulated biofilms (glycerol addition and PEG400 addition) and encapsulated black carbon (no biomass), where biofilm or black carbon was coated by individual coating of adjusted recipe-E and recipe-F. Dashed lines represent method detection levels. Recipe-A: 1:20 TEOS:water, double sinking, 1:3 sol to phosphate buffer, aging under vacuum. Gel plate recipe: 21:1 (water:TEOS) or 1:1.55:11.41 (MTES:TEOS:water), 20% (v/v) PEG400; 21:1 (water:TEOS) with 6% (v/v) glycerol or 1:1.55:11.41 (MTES:TEOS:water) with 20% (v/v) glycerol. Adjusted recipe-E: MTES/TEOS/water=0.5:0.5:20, PVP=10 mg with 20% (v/v) glycerol, single coating (tea ball); adjusted recipe-F: MTES/TEOS/water=0.5:0.5:20, PVP=10 mg with 20% (v/v) PEG400, single coating (tea ball).

FIG. 7. Table 1. Summary of each recipe change effect on gel surface of individual gel coating outcome.

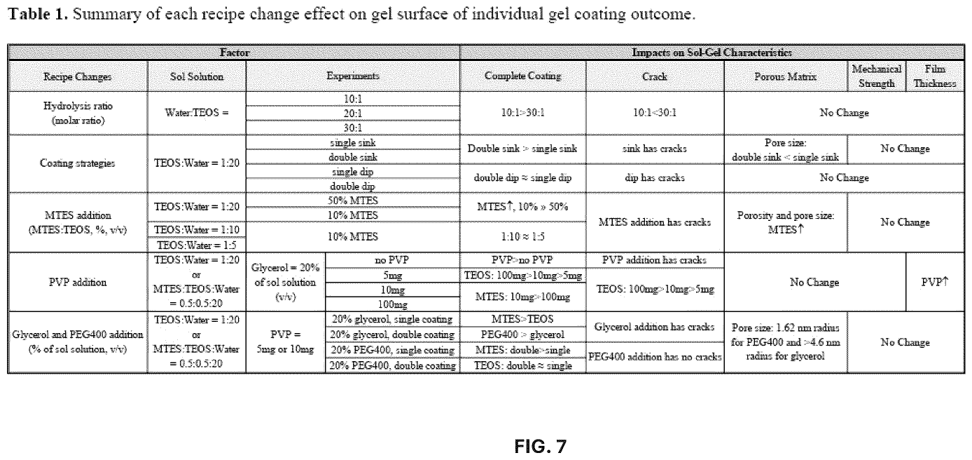

FIG. 8. Changes in PCB congener mass over time in the aqueous (column 1) and solid (column 2) phases, in the presence of uncoated and sol-gel coated LB400 biofilms on CKB: PCB4 (row 1), PCB 18 (row 2), and PCB52 (row 3). PCBs in both aqueous and solid phases were measured at each time point, where solid phase PCBs were considered sorbed to materials (sol-gel and/or CKB). All three “killed biofilm” indicate abiotic sorption controls corresponding to “benzoate soak+sol-gel_{GLY}/sol-gel_{PEG}/biofilm only”. Data points are the average of measurements from triplicate bottles, and the error bars are the standard error. Dashed lines in “aqueous phase” column represent remaining aqueous PCB mass in abiotic controls, and dashed lines in “solid phase” column represent sorbed PCB mass in abiotic controls, both of which were calculated based on K_{d} at sorption equilibrium. The differences between abiotic controls and biotic treatments were considered PCB biodegradation by biofilms.

FIG. 9. BphA expression levels (transcript per gene ratios=transcript abundance/gene abundance) of sol-gel coated and uncoated biofilms under standard conditions at day-45. Purple triangles represent “benzoate soak+no sol-gel coating” controls; black squares represent “no soak+sol-gel_{GLY}” controls; green circles represent “benzoate soak+sol-gel_{GLY}” treatments; and orange diamonds represent “benzoate soak+sol-gel_{PEG}” treatments. Black lines represent the mean of biological replicates (n=3) and error bars represent the standard error.

### DESCRIPTION

The following descriptions and examples illustrate embodiments of the present disclosure in detail. Although the present disclosure has been described in some details by way of illustration and example for purposes of clarity and understanding, it will be apparent that certain changes and modifications can be practiced within the scope of the appended claims.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment. It is to be understood that the present disclosure is not limited to the particular embodiments described herein and as such can vary. Those of skill in the art will recognize that there can be variations and modifications of the present disclosure, which can be encompassed within its scope.

All patent filings, websites, other publications, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated.

Any feature, step, element, embodiment, or aspect of the disclosure can be used in combination with any other unless specifically indicated otherwise.

The persistence of polychlorinated biphenyls (PCBs) in environmental matrices, particularly in sediments, poses significant challenges for remediation efforts. Despite being banned for decades, PCBs continue to bioaccumulate in food webs and expose nearby communities through inhalation and other pathways, leading to adverse health effects. Lower chlorinated PCBs (LC-PCBs), which are more volatile, exacerbate this issue by mobilizing from sediments into water and air, especially during dredging or through natural processes like anaerobic reductive dechlorination. While bioaugmentation using aerobic microorganisms has shown promise in degrading LC-PCBs, the stability and longevity of biofilm-based remediation systems remain a notable limitation. Biofilms grown on black carbon (BC) have demonstrated enhanced PCB-degrading activity compared to suspended cells; however, environmental stressors such as salinity, temperature fluctuations, and shear forces often lead to biomass washout and reduced biofilm viability over time, undermining the effectiveness of in situ remediation strategies.

The present disclosure addresses these limitations by introducing a novel sol-gel encapsulation approach for biofilm-enriched BCs, which significantly enhances the stability and biodegradation potential of biofilms under adverse environmental conditions. Unlike conventional methods that focus on planktonic cells or unprotected biofilms, the disclosed sol-gel matrix provides a protective, porous, and mechanically stable coating that prevents biofilm detachment and washout. The sol-gel composition, comprising tetraethyl orthosilicate (TEOS), methyltriethoxysilane (MTES), and optional additives such as glycerol, polyethylene glycol (PEG400), and polyvinylpyrrolidone (PVP), is specifically tuned to maintain biofilm integrity while allowing efficient mass transfer of contaminants to the biofilm microenvironment. This encapsulation method not only preserves cell viability for extended periods, even in the absence of external carbon sources, but also enhances the expression of degradation enzymes, such as bphA, thereby improving the overall biodegradation efficiency.

By leveraging the biocompatibility, high porosity, and mechanical strength of the sol-gel matrix, the described solution addresses the challenges of biofilm instability and limited degradation activity in harsh environmental settings. This approach provides a robust platform for in situ sediment remediation, enabling prolonged and effective degradation of PCBs and other environmental pollutants, while mitigating exposure risks to ecosystems and human populations.

### Definitions

The following definitions supplement those in the art and are directed to the current application and are not to be imputed to any related or unrelated cases, e.g., to any commonly owned patent or application. Any methods and materials similar or equivalent to those described herein can be used in the practice for testing of the present disclosure. Accordingly, the terminology used herein is for the purpose of describing particular embodiments only and is not intended to be limiting.

In this application, the use of the singular includes the plural unless specifically stated otherwise. It must be noted that, as used in the specification, the singular forms “a”, “an”, and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C”. The term “or” can be used conjunctively or disjunctively unless the context specifically refers to a disjunctive use.

Furthermore, the use of the term “including” as well as other forms, such as “include”, “includes” and “included”, is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, such as within 5-fold, and within 2-fold, of a value. Where particular values are described in the application and claims, unless otherwise stated, the term “about” meaning within an acceptable error range for the particular value should be assumed.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

### Biofilm-Based Bioremediation

The rapid growth of chemical industries and anthropogenic activities has resulted in the global contamination of soil, sediment, and aquatic environments, and this has become a significant issue of concern. Long-term persistence and accumulation of environmentally recalcitrant pollutants, including polycyclic aromatic hydrocarbons (PAHs), chlorinated compounds, pesticides, phthalates, and inorganic heavy metals ions, has altered the nature of the environment and poses a risk of contamination of environmental matrices, leading to various hazardous health issues for living organisms. PAH compounds such as naphthalene, anthracene, benzo (o) pyrene, and phenanthrene are well-known persistent organic pollutants (POPs), which are widely distributed in the environment due to the incomplete combustion of organic matter. They are adsorbed onto soil particles. These compounds have shown moderate to high acute toxicity and have mutagenic, carcinogenic, teratogenic, and immunotoxic effects on living organisms, including humans, animals, and microorganisms.

Increased concentrations of toxic metals also represent a threat to public health and natural biota. Some heavy metals, such as chromium (Cr), copper (Cu), arsenic (As), mercury (Hg), and lead (Pb) have a high degree of toxicity. These metals can cause severe health consequences such as neuromuscular defects, growth and developmental abnormalities, mental illness, skin damage, cardiovascular diseases, kidney dysfunction, hearing sickness, and cancer. Heavy metals can also cause significant modifications and alterations in cell signaling and protein folding, inhibition of enzymatic reactions, and other physiological functions. High-dose metal exposure, particularly Cd and Pb, may induce oxidative stress, enzyme inactivation, and suppression of the antioxidant defense. The metals can also interact with cell components such as DNA and proteins, resulting in DNA damage and conformational changes.

Pesticides like organophosphates, carbamates, and pyrethroids are the most common, and ~2.4 million metric tons of pesticides have been used worldwide to control various insects, pests, weeds, and other unwanted organisms in the agricultural, household, commercial, and industrial sectors. Pesticide residues can persist in the environment for many years and can be detected from very low to higher concentrations in soil and water systems. Excessive human exposure to pesticides at a relatively high concentration can cause carcinogenicity, genotoxicity, cytotoxicity, neurotoxicity, and fertility disorders.

Due to these environmental toxins, removal of such toxic contaminants from the environment in an ecofriendly manner is needed. Bioremediation has developed as an inexpensive, environmentally safe, and suitable treatment technology in the biodegradation of organic and inorganic pollutants in contaminated areas. Bioremediation uses the metabolic capabilities of a wide range of microbial inoculants for the alleviation and amelioration of environmental pollutants. Based on the treatment methodology and presence of contaminants, various methods of in situ and ex situ bioremediation for the efficient reduction and removal of hazardous pollutants are available (in situ bioremediation offers onsite treatment, while ex situ provides offsite treatment of the contaminated pollutant).

Biofilm-based bioremediation technology can be used for the removal of pollutants, such as those found in contaminated groundwater and soil. Bioremediation is an eco-friendly, sustainable technology and cost-effective tool that uses microbes to remove contaminants from soil and water along with the decomposition of a wide variety of pollutants into comparatively less harmful substances. The microbial groups of biofilms are able to successfully remediate the contaminants from soil and water, such as heavy metals, petroleum products, explosives, pesticides, and insecticides.

Several bacterial strains have the ability to colonize and secrete biofilms. Many bacterial species, such as Bacillus, Rhodococcus, Arthrobacter, Cycloclasticus, and Alcanivorex, are biofilm-forming bacteria and exhibit capacity to degrade or mineralize hazardous pollutants in various contaminated environments. Biofilm applications have beneficial uses areas like bioremediation (cleaning up pollutants, such as oil spills), wastewater treatment, improving water quality, microbial fuel cells, industrial processes like corrosion control, food production (probiotics), medical applications (wound healing), and environmental monitoring. Examples of beneficial biofilms include but are not limited to: biofilms created by Alcanivorax borkumensis, which used hydrocarbons as food source, such as those found in toxic oil spills, as well, Cycloclasticus species also breakdown/clean up toxic chemicals, such as those found in oil spills and biofilms created by Paraburkholderia xenovorans LB400 can be used for degradation of wide ranges of PCBs (mono- to hexachlorinated biphenyls).

Applications of the invention also include are bioreactors, fixed-film bioreactors (e.g., rotating biological contactors), tricking filter, moving bed bioreactor, such as those used in municipal and industrial wastewater treatment, but can also be used in other applications such as treating perchlorate in groundwater.

Biofilms can also be engineered through biotechnological techniques by manipulating regulatory networks and regulating gene expression to enhance their performance in order to completely degrade toxic pollutants and halt their exposure in the environment. Advances in synthetic biology have enabled the reprogramming of biofilms to improve their function and enhance their biodegradation capability. In recent years, genetic engineering has enabled the successful application of biofilms as biocatalysts in biotransformation processes. Li et al. (2018) constructed a genetically modified Bacillus subtilis strain, N4/pHTnha-ami, which showed complete degradation of organonitriles from wastewater. Moreover, J. Huang et al. (2019) constructed B. subtilis biofilm that was capable of degrading highly toxic mono (2-hydroxyethyl) terephthalic acid to less toxic terephthalic acid. Embodiments provide recombinant bacteria for forming beneficial biofilms.

### Feedstock

Black carbon (BC), such as activated carbon or biochar is a stable solid, rich in carbon, that is usually made from organic waste material or biomass that is partially combusted in the presence of limited oxygen (a process known as pyrolysis). The qualities that make up black carbon vary depending upon the material that it comes from (feedstocks, i.e., trees (such as forestry cuttings, wood chips, sawdust, leaf litter, bamboo, wood, timber slash), energy crops (e.g., straw, corn cobs, corn stalks, corn kernel (small or large), stover, rice husks, bagasse), nut shells (e.g., coffee bean shells, walnut shells, cashew nut shells, pistachio shells, coconut hulls), grass, animal materials (poultry litter, manure (pig or dairy), etc.). In some embodiments, corn kernel biochar is used to grow biofilms.

### Sol-Gel

Currently, application of sol-gel is only focusing on viable whole cells and biomolecules, but provided herein is the discovery and use for biofilm integrity protection. Biofilm-enriched BCs can be used in, for example, sediment remediation to degrade toxic contaminants (e.g., lower chlorinated PCBs) and mitigate air flux. Nevertheless, the biomass abundance of bio-augmenting cells (e.g., PCB-degraders) with BCs decreases over time after application for in-situ sediment remediation. Provided herein is the protection of enriched biofilms to increase the longevity of contaminant biodegradation activity.

Functional biofilms on BC are efficient at removing contaminants because biofilm formation and BC feedstock can induce functional gene expression, as has been demonstrated. However, unfavorable environmental conditions (salinity, shear force) can interfere biofilm stability and affect biodegradation potential. Provided herein is a specifically tuned sol-gel for biofilm encapsulation which can help maintain biofilm against adverse environmental conditions and cell washout by providing a protective layer outside the biofilm surface. Sol-gel has been used to protect planktonic cell function because sol-gel is biocompatible and highly porous, providing mechanical strength, as well as chemical, thermal, and radiant stability, but it has never been used for biofilms. Thus, provided herein is a novel sol-gel application on biofilms to prolong the biodegradation potential. In an example, PCB-degrading biofilm and corn kernel biochar is used as a model to develop sol-gel coating process. This sol-gel method provides platform technology for various remediation applications.

The sol-gel process is a method for producing solid materials from small molecules. The method is used for the fabrication of metal oxides, especially the oxides of silicon (Si) and titanium (Ti). The process involves conversion of monomers in solution into a colloidal solution (sol) that acts as the precursor for an integrated network (or gel) of either discrete particles or network polymers.

In embodiments, sol-gel can be used to form thin films, which can be produced by, for example, spin coating, dip coating, spraying electrophoresis or roll coating. It can also be cast in mold. This protective coating can be applied to composition comprising biofilm and feedstock.

In some embodiments, the sol gel is made from metal oxides, such as aluminates, titanates, and/or zirconates; alkoxysilanes (silicon alkoxides), such as etramethoxysilane (TMOS) and tetraethoxysilane (TEOS); silica and silicate glasses and coatings (SiO₂-based); Other metal oxides such as alumina (Al₂O₃), titania (TiO₂), zirconia (ZrO₂), and related mixed oxides and ceramics are also made by sol-gel routes. The sol gels can also include other components, including but not limited to, water, alcohols (e.g., ethanol, methanol), acids or bases to adjust poH, surfactants, block copolymers or foams, as well as glycerol and/or polyethylene glycol (PEG), such PEG200, PEG400 or PEG600.

In some embodiments, the sol gel is made from TEOS and optionally includes MTES.

### TEOS or TMOS

Tetraethyl orthosilicate (TEOS) is the organic chemical compound with the formula Si(OC₂H₅)₄. TEOS is a colorless liquid. It degrades in water. TEOS is the ethyl ester of orthosilicic acid, Si(OH)₄. It is the most prevalent alkoxide of silicon.

TEOS easily converts to silicon dioxide upon the addition of water: Si(OC₂H₅)₄+2 H₂O→SiO₂+4 C₂H₅OH This hydrolysis reaction is an example of a sol-gel process.

Also for use herein is tetramethylsilane (TMOS)—Si(CH₃)₄.

### MTES

Methyltrimethoxysilane (MTES) is an organosilicon compound with the formula CH₃Si(OCH₃)₃. It is a colorless, free-flowing liquid. It is a crosslinker in the preparation of polysiloxane polymers.

In some embodiments, the sol-gel further includes glycerol and/or polyethylene glycol (PEG), such PEG200, PEG400 or PEG600.

The following non-limiting example is provided to further illustrate the present invention.

### Example

This example is provided for illustrative purposes only and not to limit the scope of the claims provided herein.

### Introduction

There is a need for additional biofilm protection to increase the longevity of, for example, PCB biodegradation activity.

Encapsulating biofilms with sol-gel holds promise to enhance sustained cell viability and promote prolonged degradation activity. The sol-gel matrix is biocompatible and highly porous, providing mechanical strength and chemical stability. (20-22) These characteristics make sol-gel a robust framework to enable transport of substrates and protect biomass against environmental changes. Silica gel is increasingly being used to encapsulate viable whole cells and biomolecules (e.g., protein and enzymes) (23-25), and encapsulation has been shown to increase enzyme activity and bacterial degradation performance. (22, 25, 26) A common two-step process is used to produce sol-gel as follows (27): a polymer-forming precursor (e.g., TEOS (tetraethyl orthosilicate), sodium silicate) is hydrolyzed in an acid/base catalyst until sol solution reaches hydrolysis and condensation equilibrium; (20, 21, 24, 28, 29) condensation and gelation are accelerated by adding acid/base catalyst, and gel becomes dense and cross-linked with aging. The characteristics of the sol-gel hold high potential and feasibility for application on biofilm-enriched BCs, yet sol-gel has not been used to encapsulate and protect biofilms.

The versatility of sol-gel chemistry allows tuning of coating characteristics (e.g., mechanical properties, gel microstructure, and cell viability) for numerous applications. For example, addition of MTES (methyltriethoxysilane) to TEOS in sol solution can adjust the hydrophobicity of the final gel product and thereby affect mass transport of substrate. (30) The use of additives, such as glycerol and polyethylene glycol (PEG), and/or the expulsion of cytotoxic hydrolysis byproducts (e.g., alcohol) during encapsulation, increase gel pore size and improve cell viability. (20, 31-33) Polyvinylpyrrolidone (PVP) helps the applied coating layer to adhere better over the hydrophobic core, while avoiding micro-cracks. (34-36) Thus, adjusting of sol-gel chemical conditions can tune properties for specific applications. Employing sol-gel to protect biofilms should maintain cell viability.

Provided herein is the development of a novel sol-gel encapsulation approach for coating aerobic PCB-degrading biofilm-enriched biochar. Recent work discovered that BC feedstocks are used in influencing biofilm formation and gene expression to improve PCB biodegradation potential. (16) To further advance in-situ remediation applications, protecting the biofilm against adverse environmental conditions is needed for deployment. In addition to PCB, this sol-gel approach can also serve as a platform technology for a suite of potential bioremediation applications. It is hypothesized that biofilms grown on biochar encapsulated with sol-gel would increase the longevity of the biofilm's viability and activity by providing bacteria with a favorable environment. This needed to be tested as sol-gel can be toxic. To test the hypothesis, the sol-gel coating chemical recipe was developed and adjusted, physical properties (porous structure, observational hardness, and surface coverage) of the produced sol-gel were measured, and encapsulated biofilm viability and PCB degradation potential was evaluated. It was discovered that this novel encapsulation approach protects biofilms against adverse environmental changes (high salinity and continuous shear force) and extended cell viability for over three months without additional carbon sources.

### Materials and Methods

Chemicals. TEOS and MTES liquid solutions were purchased from Acros Organics (Antwerpen, Belgium). Corn kernel biochar was selected to grow biofilms because it was previously demonstrated that this type of BC improved cell attachment and functional gene expression; details of biochar characteristics can be found in prior work. (16) Corn kernel biochar was obtained from Dr. Albert Ratner's research group at UIowa. (37) Glycerol and PEG400 were purchased from Sigma-Aldrich (Burlington, MA) and TCI America (Portland, OR), respectively. PVP was purchased from MP Biomedicals (Santa Ana, California).

Experimental design. The desired encapsulation outcome is complete coating, a porous matrix, and robust observational hardness. Complete coating aims to protect biofilms/cells from escaping to maintain biomass. A porous matrix can allow efficient mass transfer of nutrients/pollutants between the surrounding environment and inside sol-gel, and concurrently decrease cell leaching. High observational hardness helps prevent the damage of sol-gel coating from shear force, dissolution, and breaking. All of these are designed to have stable sol-gel structure, adequate food/energy supplies, and extend cell survival and activity in a real environment.

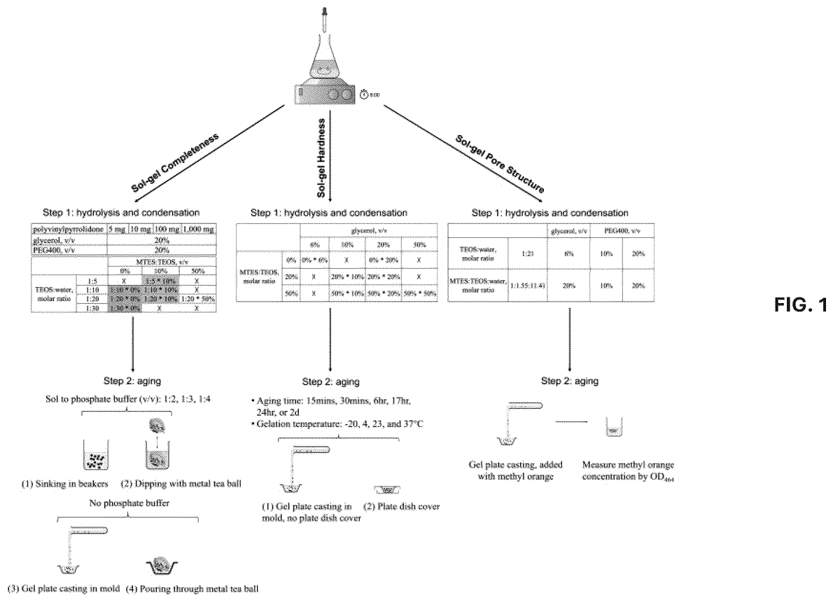

To achieve the desired encapsulation, multiple parameters were systematically tested for their impact on sol-gel completeness, hardness, and pore structure (FIG. 1). For complete coating, a series of factors were evaluated, including phosphate buffer, hydrolysis ratio, coating strategies, MTES addition, PVP addition, and glycerol/PEG400 addition. For gel hardness, the impacts of aging temperature, phosphate buffer, MTES and glycerol addition, aging time, and dish cover were compared. Lastly, the effect of adding different quantities of glycerol and PEG400 was investigated for controlling pore structure. It was predicted that MTES addition, PVP addition, lower hydrolysis ratio, and a double coating would result in a more cohesive gel surface; more MTES, longer aging time, and dish cover would yield a harder gel; and that more glycerol or PEG400 would increase pore size.

### Formulation of Various Precursor-Derived Gels.

Tetraethyl orthosilicate. TEOS was mixed with water in molar hydrolysis ratio of 20:1 (water to TEOS), and an acid catalyst was applied by adding 1 mol/L HCl to sol solution until pH=1 to increase the hydrolysis rate of alkoxysilanes. (27) The solution was continuously mixed on the platform shaker in the fume hood with open head space to allow complete hydrolysis and alcohol evaporation (at least 4 hours). A strong base, 1 mol/L KOH, was added to adjust the sol solution to a neutral pH. BC in phosphate buffer (35 mM; pH 8.0) was added into neutral sol solution in a ratio of 1:3 (v/v, sol solution: BC in phosphate buffer). Gelation occurred in 5 minutes at room temperature, and the final pH was about 8. This entire process is referred as the “TEOS original recipe” below.

### Assessment of Encapsulation Recipe Variation Impacts on Sol-Gel Completeness.

Gel Imaging. Coating completeness and pore structure (pore size and porosity) of coated BCs made with each recipe adjustment were visualized by Scanning Electron Microscopy (SEM) using Hitachi S-4800 (Tokyo, Japan).

Phosphate buffer to sol solution ratio. Phosphate buffer was employed to help stabilize pH of neutralized sol solution (buffer between pH 5.8 and pH 8.0) and avoid damaging cell viability during gelation. Thus, various ratios of sol solution to phosphate buffer containing BC were compared to evaluate impacts on the coating completeness. Nevertheless, the sol solution water content could increase gelation time and potentially interfere with coating performance; therefore, BC without phosphate buffer was also compared in treatment groups. Ratios of sol solution to phosphate buffer varied from no phosphate buffer, 1:2, 1:3, and 1:4 (v/v). Other steps were the same as in the TEOS original recipe.

Coating strategies. A variety of coating strategies were applied to encapsulate BCs with neutral sol solution to make coating more complete. (1) Sinking in beakers (single coating vs double coating). A phosphate buffer with BC was poured into a beaker with neutralized sol solution, then was aged for one day. BCs were coated twice by repeating the same process. (2) Dipping with metal tea ball (single coating vs double coating). Sol solution was first neutralized in a beaker with phosphate buffer, and then a metal tea ball holding BC was dipped in the neutral sol solution and dried for 2-5 minutes. The dip-dry-dip coating was repeated until the sol solution in beaker became gelated, and then the coated BCs were aged in the fume hood for a day. This entire process was repeated one additional time following aging. (3) Gel plate casting in mold. BC without phosphate buffer was directly added to neutralized sol solution, fully vortexed for a minute and poured to mold for casting and aging. Waxed paper with cylinder-shaped mold and hydrophobic plastic weighing boats were used as molds because both have non-adhesive surfaces that allow easy removal of the sol-gel product. The resulting product was BCs randomly distributed within the regular shape of a gel plate. (4) Pouring through metal tea ball. BCs without a phosphate buffer were added to neutralized sol solution, fully vortexed for a minute and then immediately poured into the tea ball. The filtrate sol solution was repeatedly poured onto the BCs surface until gelation. The resulting product was individual BCs coated separately with the tea ball filtering redundant sol solution.

Hydrolysis ratio. Before condensation, guaranteeing complete hydrolysis requires a hydrolysis ratio of water over silicon alkoxide to be >four. (38) To evaluate the impacts of the hydrolysis ratio on encapsulation completeness, TEOS was mixed with water in a series of incremental hydrolysis ratios (water:TEOS) to make sol solution: 10:1, 20:1, and 30:1. Other steps were the same as in the TEOS original recipe.

MTES addition. Adding MTES could control the growth of silica particle size and thus improve sol-gel surface cohesiveness. (30) To evaluate the impacts of MTES addition on sol-gel coating completeness, sol solution was mixed at a hydrolysis ratio of 20:1 (water to TEOS) in addition to two MTES volumes: 10% and 50% (MTES:TOES, v/v). Different hydrolysis ratios of water to TEOS (10:1 and 5:1) were also added to 10% (MTES:TOES, v/v) MTES to check coating completeness. Other steps were the same as in the TEOS original recipe.

Polyvinylpyrrolidone addition. To evaluate whether adding PVP aided sol-gel coating completeness and cohesive smooth surface formation, different masses of PVP were added to sol solution prior to shaking for hydrolysis and ethanol evaporation: 1 g, 100 mg, 10 mg, or 5 mg. PVP was first dissolved in 12 mL DI water and then added to either 20:1 (water:TEOS) or 0.5:0.5:20 (MTES:TEOS:water). BCs without phosphate buffer were directly added to neutralized sol solution containing 20% glycerol (v/v), fully vortexed for a minute and poured into the tea ball immediately. The other steps were the same as the TEOS original recipe.

Glycerol and PEG400 addition. To test the impacts of glycerol and PEG400 on gel coating completeness, 20% glycerol or PEG400 was added to sol solution (v/v), followed by pouring neutralized BC (no phosphate buffer) through a tea ball. Sol solution was mixed by either TEOS:water=1:20 or MTES:TEOS:water=0.5:0.5:20, together with 5 mg or 10 mg PVP added. The coating process was conducted twice; other steps were the same as the TEOS original recipe.

Assessment of encapsulation recipe variation impacts on sol-gel (gel plate) hardness. The hardness of the gel plate was evaluated by controlling gelation temperature (−20, 4, 23, and 37° C.), MTES addition (20% or 50% molar of TEOS), glycerol addition (6%, 10%, 20%, or 50% of sol solution, v/v), aging time (15 mins, 30 mins, 6 hr, 17 hr, 24 hr, or 2 d), location (plate dish cover or not), and hydrolysis ratios (1:20 or 1:4, TEOS:water, v/v). Hardness of gel plate was qualitatively evaluated by observational methods. Relative hardness was compared by applying consistent pressure onto the surface of the gel plate products and visually inspecting the resulting deformations (e.g., cracks on the gel plate surface). Additionally, hardness was also tested by placing gel plates in K1 medium and applying continuous shaking at 150 rpm to observe the gel plate resistance to wear. The visual inspection approaches, albeit non-quantitative, were important for assessing the practical durability of the gel plates.

Sol-gel porosity and pore size adjustment by glycerol and PEG400. As described above, glycerol or PEG400 was added to the neutralized sol together with MTES. Aside from checking impacts on sol-gel observational hardness and completeness, the addition of glycerol or PEG400 was also intended to control the sol-gel's pore size and porosity by decreasing surface tension and osmotic stress on microbes. A desirable pore size should be larger than the target contaminant molecule size and smaller than the biodegrading bacterial size. Thus, different portions of glycerol or PEG400 were added to the neutralized sol to compare with no glycerol or PEG400 addition. 6% glycerol, 10% PEG400, and 20% PEG400 (glycerol/PEG400: sol, v/v) were added in sol solution of 21:1 (water:TEOS) hydrolysis ratio; 20% glycerol, 10% PEG400, and 20% PEG400 (glycerol/PEG400: sol, v/v) were added in sol solution of 1:1.55:11.41 (MTES:TEOS:water) hydrolysis ratio. To check pore size, methyl orange (0.76 g/L), with 6-8 nm molecular diameter, (39) was added to neutralized sol solution in 0.5% of total sol solution volume. Aged sol-gel was added to DI water and OD464 of the solution was measured with Varian Cary 50 Bio UV-Visible Spectrophotometry over time.

Encapsulation of biofilm-enriched BCs with sol-gel using developed recipes (complete, porous, and highly mechanical stable coating). Methods for growing Paraburkholderia xenovorans LB400 (LB400) on BC can be found in prior work. (16) Briefly, LB400 cells were grown in K1 medium (250 mL) containing biphenyl crystal (0.19 g) until cell cultures reached mid-exponential phase (OD600-0.5-0.6), then LB400 concentrated via centrifugation (3 mL, OD600=1) were resuspended in K1 medium (27 mL) containing corn kernel biochar (0.9 g) and biphenyl crystal (22.8 mg) for 10-days of growth. LB400 biofilm-BC was encapsulated with the tea ball approach to form individually coated biofilm-BCs. Sol solution was first prepared by mixing at a hydrolysis ratio of 1:20 (TEOS:water) or 0.5:0.5:20 (MTES:TEOS:water), with 10 mg of PVP added, followed by shaking at 200 rpm in a fume hood for 6-7 hours. Sol solution was then neutralized by 1 mol/L KOH and 20% autoclaved glycerol or PEG400 (v/v) in a 15-mL centrifuge tube, vortexed for one minute, and then biofilm-BCs were added into the tube. The biofilm-BCs together with sol solution were fully vortexed for 10-15 s and poured into the autoclaved tea ball immediately. Residual sol solution was collected using the weigh boat (wiped with 70% ethanol) and repeatedly poured onto biofilm-BCs until gelation occurred. Coated biofilm-BCs were aged for one day in a sterile beaker and then the entire coating processes were repeated for the double-coated biofilm-BCs.

The process of sol-gel coating LB400 biofilm-BCs also occurred in the gel plate. Sol solution was first mixed at the hydrolysis ratio of 21:1 (water:TEOS) or 1:1.55:11.41 (MTES:TEOS:water), by shaking in the fume hood at 200 rpm for 6-7 hours. Each sol solution was neutralized with 1M KOH and autoclaved glycerol (6% or 20%, v/v) or PEG400 (20%, v/v) in a 15-mL centrifuge tube and vortexed for one minute before biofilm-BCs were added into the tube. The biofilm-BCs together with sol solution were fully vortexed for 10-15 s and then immediately poured onto the weighing boat (wiped with 70% ethanol). Coated biofilm-BCs were aged under a sterile plate dish; gel plates made with TEOS-only sol solution were aged for <19 hrs while gel plates made with MTES addition were aged for <1 hr.

Evaluation of coated-biofilms viability and biodegradation potential. To investigate the performance of sol-gel protecting biofilm viability and integrity, live and dead cell distributions of LB400 biofilms on BCs were compared between non-coated biofilms and coated biofilms made from different recipes. Coated and non-coated biofilms were treated in either three-round of reuse (10 mM benzoate in each round; biofilms were transferred to fresh media for reuse) for 25 days or carbon source starving conditions (only one time 10 mM benzoate addition) for 3 months. The live/dead cell distribution before and after treatment was stained with SYTO9 and propidium iodide and then observed by Confocal Laser Scanning Microscopy (CLSM) with the Leica SP8 STED Super Resolution Confocal (Leica Microsystems, Exton, PA). Details of CLSM methods can be found in prior work. (16)

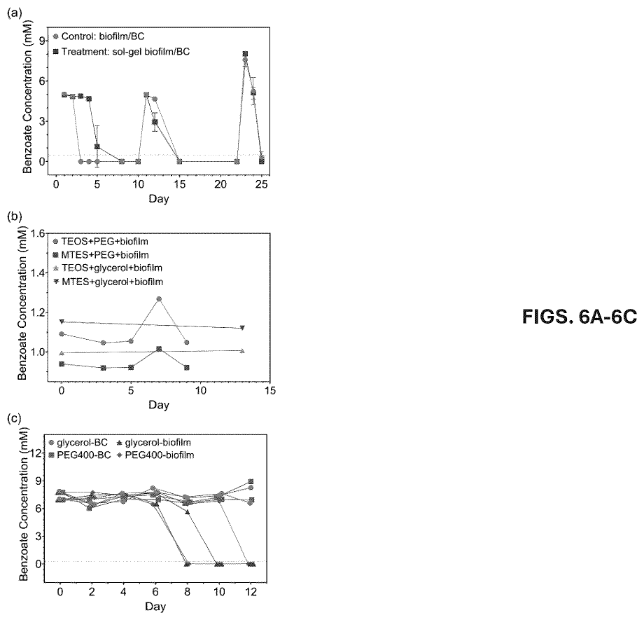

To evaluate the biodegradation potential of sol-gel coated biofilm-BCs, both individually coated biofilm-BCs and gel plate coated biofilm-BCs made with various recipes (details in FIG. 6) were added to K1 medium containing benzoate (10 mM) or acetate (3 mM) to track biodegradation. Benzoate and acetate were chosen as model chemicals to test the activity of coated cells and biofilms because both are less sorptive, more soluble, and thus more easily measured than PCBs in the aqueous phase. (16) Benzoate and acetate concentrations were quantified over time using high performance liquid chromatography (Agilent 1100 series) and Ion Chromatography (Dionex ICS-2100), respectively. Details of carbon source measurement can be found prior work. (16)

Test of environmental condition impacts on individually coated BCs. To evaluate the impacts of both salinity and shear force on sol-gel surface integrity, 0.06 g of individually coated BCs were added to 20 mL of either high salinity solution (30 g/L sea salt in DI water, pH=7) or K1 medium for continuous shaking at 150 rpm for one month. Individually coated BCs were made with double coating at a hydrolysis ratio of 0.5:0.5:20 (MTES:TEOS:water) with the addition of 10 mg PVP, followed by sol solution mixing with 20% (v/v) of glycerol or PEG400. Sol-gel surface was visualized for comparing before and after one-month test using SEM imaging. The Brunauer-Emmett-Teller (BET) surface area and pore size distribution were measured by using Quantachrome Nova 4200e, and the data collected were analyzed by the NovaWin software.

Statistical analysis. Statistical analysis was conducted in GraphPad Prism 9 and Microsoft Excel. Normality of experimental data was tested by Shapiro-Wilk and Kolmogorov-Smirnov tests and establishing normal and log-normal probability plots. A pairwise two-sided t-test or one-way ANOVA was applied to test for differences between treatment means depending on experimental design. Differences were considered significant at 95% confidence level (alpha=0.05).

### Results and Discussion

### Characterization of the Optimal Coating Recipe.

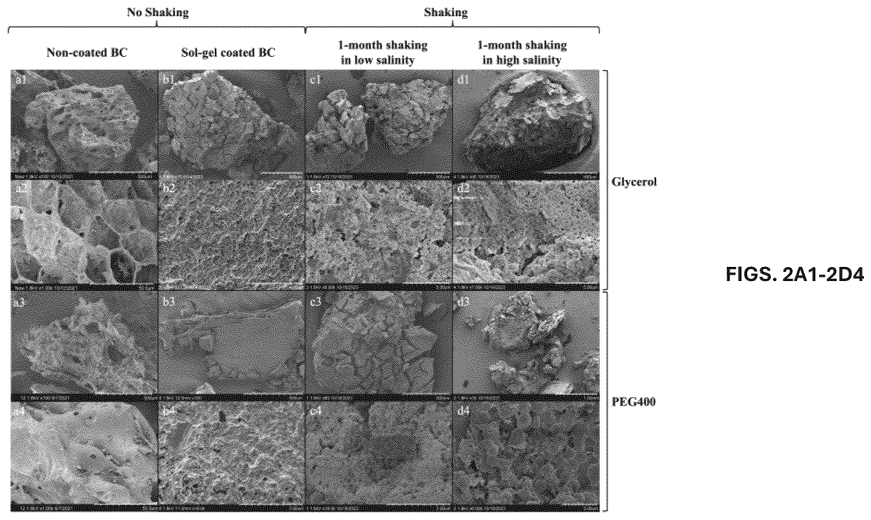

Based on the encapsulation goals, a recipe was developed for coating BCs with desired sol-gel properties after iterative adjustment of chemical compositions and coating strategies. Briefly, the sol solution MTES/TEOS/water/PVP (molar ratio=0.5:0.5:20:7.7×10⁻⁶) at pH=1 was mixed for 6-7 hours, and 20% (v/v) of glycerol (MTDP-G) or PEG400 (MTDP-P) were vortexed, followed by KOH neutralization to pH=7. BCs or biofilm-enriched BCs were added and filtered through a tea ball. Compared to non-coated BCs (FIG. 2A), the BC surface was fully coated by sol-gel after aging (FIG. 2B), and the sol-gel surface exhibited pore sizes (>4.6 nm radius for MTDP-G and 1.62 nm radius for MTDP-P) smaller than bacteria and larger than nutrient/pollutant molecules. In addition, the sol-gel structure was not damaged by shear force and high salinity after encapsulated BCs was shaken in both low salinity (K1) and high salinity (30 g/L sea salt in DI water) for one month (FIGS. 2C and 2D). This indicates that both sol-gel recipes MTDP-G and MTDP-P yield encapsulated BCs with continuous, complete, porous, and high observational hardness surfaces.

### Impacts of Coating Parameters on Sol-Gel Completeness.

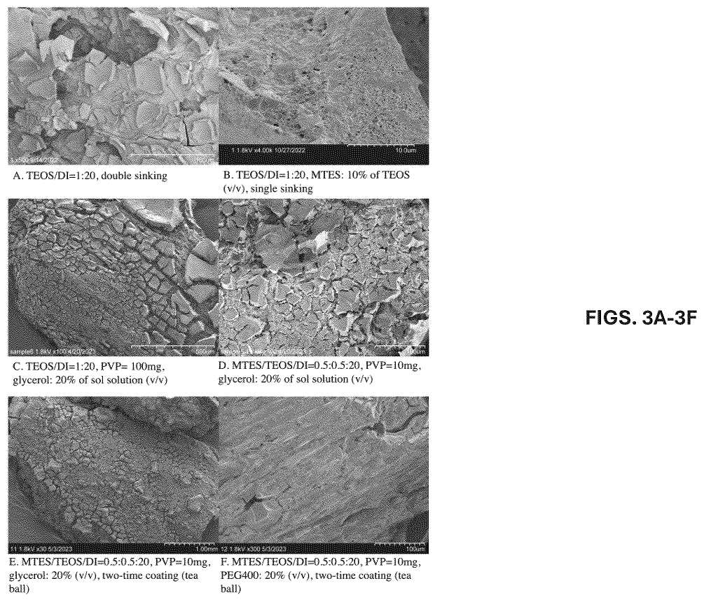

Hydrolysis ratio: Adjusting the hydrolysis ratio (water:TEOS) alone improved coating completeness but a complete sol-gel coating did not develop on BC surfaces even though theoretical complete hydrolysis was achieved in the sol solution. Through SEM images of coated BCs (FIG. 3A), it was observed that the lower hydrolysis ratio (10:1) formed more complete coating and fewer cracks on sol-gel surfaces than the higher hydrolysis ratio (30:1); however, all three ratios yielded incomplete coatings. More BC surfaces were covered by gel made under lower hydrolysis ratios (10:1, 20:1), potentially because increasing hydrolysis ratio promotes siloxane bond cleavage reactions in the gel. (27) Therefore, lower hydrolysis ratios were selected for later testing.

Coating strategies: To address the incomplete coating issues, various coating strategies on sol-gel surface coverage completeness were evaluated. The coating performance was compared between sinking in beakers and dipping with tea ball approaches, as well as between single- and double-coating. It was found that there were sol-gel surface cracks using both sinking and dipping approaches. Dipping once or twice yielded no difference in coating completeness, which was potentially due to less sol-gel contact between BCs and sol solution, while double sinking resulted in more coated surfaces and smaller pore sizes (generally 5-7 μm, some ~200 μm) compared to single sinking although overall coating was incomplete.

MTES addition: Addition of MTES potentially inhibits SiO₂ growth and thus mitigates sol-gel cracking problems, although there were many possible causes of the sol-gel surface cracks. When the size of silica particles becomes large, the gel can reportedly crack and spall. (30) Thus, MTES was added in an effort to control particle size growth and concurrently adjust sol solution hydrophobicity, which could also affect pore size. (30) Another way to avoid large silica particles is to control the current sol solution reaction time to between 4-24 h; here it was found that when using MTES, the sol solution shaking time should be <7 hrs before the gel solidified. MTES addition to the sol solution improved the BC surface coverage by sol-gel. Some small BC pieces had the desired smooth sol-gel coating surface, high porosity, and small pore size (FIG. 3B). Nevertheless, a higher percentage of MTES addition (50%) or lower hydrolysis ratio (1:5) showed little difference regarding completeness and evenness compared to adding a lower percentage of MTES (10%) or higher hydrolysis ratios (1:10).

PVP addition: PVP was added in sol solution to improve film-forming thickness and cohesive sol-gel coating. PVP is reportedly useful in decreasing the tendency to crack, improving adhesion over the hydrophobic core, and manipulating pore size in the porous film. (34-36) Multiple masses of PVP addition to sol solution were tested: 100, 10, and 5 mg. It was found that the PVP impacts on gel completeness were related to sol solution hydrophobicity. When the sol solution was more hydrophobic (e.g., sol solution+MTES), less PVP (10 mg) was superior in terms of coating completeness and evenness compared to 100 mg of PVP (FIG. 3D). When the sol solution was more hydrophilic (e.g., sol solution with only TEOS), higher PVP addition (100 mg) yielded a complete gel structure (FIG. 3C), while most BC surfaces remained uncovered with 5-10 mg of PVP addition. Compared with no PVP addition, the sol-gel surface with PVP addition formed a thicker film and more coating regardless of coating strategies, although the PVP did not resolve gel surface cracks.

Glycerol and PEG addition: Glycerol and PEG enhanced sol-gel coating completeness and adjusted gel pore size. Both glycerol and PEG were used to increase pore size by reducing surface tension thus decreasing capillary forces during gelation and increasing biocompatibility by reducing osmotic stress. (20) Based on the results of gel plate pore size adjustments, the details of which will be presented later, 20% (v/v) of glycerol or PEG400 was added to test their impacts on sol-gel pore size and coating performance, together with effects of single- or double-coating. SEM imaging shows that sol-gel (+PEG400) had little to no gel surface cracks and mitigated gel surface peel-off compared to sol-gel (+glycerol) (FIGS. 3E, 3F). With the addition of glycerol or PEG400, sol-gel with MTES addition generated a more complete coating than only TEOS, and double coating improved coating completeness, cracks, and evenness of MTES addition gel surface compared to single coating but did not benefit the only TEOS recipe. A summary of the impacts of all coating parameters on sol-gel characteristics of individual coating is listed in Table 1.

In summary, the sol solution recipe with MTES and 10 mg PVP (MTES/TEOS/water/PVP=0.5:0.5:20:7.7×10⁻⁶), together with 20% (v/v) PEG400 and double coating, yielded the coating performance with the least cracks and the most smooth, even, and complete sol-gel surfaces. Using the same coating procedures with 20% glycerol also yielded a desirable overall sol-gel structure, although there were surface cracks. Both PEG400 and glycerol addition resulted in pore sizes (1.62 nm radius for PEG400 and >4.6 nm radius for glycerol) larger than the target pollutant molecule and smaller than LB400 cells.

Phosphate buffer to sol solution ratio: Phosphate buffer addition did not improve sol-gel coating completeness. Phosphate buffer, which can help maintain pH values between 5.8 and 8.0, may aid stability of the neutralized sol solution and thereby protect cell viability during gelation. Thus, the impacts of phosphate buffer to sol solution volume ratios (2:1, 3:1, 4:1, v/v) was tested on cell/biofilms and aging time (minutes to days) on gel status (wet to fully dried) and coating completeness (cell leaching). It was discovered that a higher cell to sol ratio required longer aging time to completely dry gels because more water needed to evaporate. Nevertheless, cell leaching and growth in the liquid solution containing carbon sources (acetate and benzoate) was observed, which indicated potentially incomplete cell coating when adding phosphate buffer in recipe.

Coating completeness of gel plate: To address the issues of coating completeness and cracking, it was attempted to encapsulate BCs with a gel plate approach. Making a gel plate requires a casting or molding process; thus, multiple casting variables were evaluated, including mold selection and phosphate buffer. A plastic weighing boat proved a superior mold compared to wax paper because the weighing boat can form a regular shape without damage. No phosphate buffer addition in sol solution could improve coating completeness and the observed hardness of the sol-gel compared to phosphate buffer addition. Specifically, the gel made with phosphate buffer hardened slowly and did not adhere well to BCs, while the no phosphate buffer gel decreased the gelation time from hours to minutes and could completely coat BCs. The ideal optimized product was permeable with propidium iodide, indicating that chemical transfer within sol-gel occurs. In general, this feasible gel plate development method completely encapsulates all BCs within sol-gel and solves cracking concerns by adding BCs to neutralized sol solution without a phosphate buffer, followed by gelation in a plastic weighing boat at room temperature or higher.

Impacts on gel plate hardness: The effects of wide ranges of temperature (−20, 4, 23, 37° C.) were tested on gel formation during gelation and it was found that higher temperature promotes formation of higher observational hardness gels. Thus, 23° C. was chosen for remaining experiments based on convenience and relatively high gel hardness. Although the gel plate product did not dissolve in either DI water or high salinity (40 g/L) solution for at least two weeks, coated BC particles could escape from sol-gel plate after two days shaking at 150 rpm because the gel partially broke down due to intense shear force. This result was undesirable from an experimental perspective because such disintegration complicates determining if pollutant biodegradation was a result of encapsulated BCs or partially by escaped BCs; furthermore, disintegration under shear would also be undesirable for field deployment. Thus, the observed hardness of sol-gel was controlled by incorporating MTES to avoid potential BC escapes from the gel plate. The impacts of varying MTES additions (20% and 50% molar ratio, MTES/TEOS) and glycerol (10%, 20%, and 50% glycerol/sol solution, v/v) was tested. When combining these two chemical adjustments, the observational hardness ranked as follows: 10% glycerol+20% MTES>20% glycerol+50% MTES>20% glycerol+20% MTES or 10% glycerol+50% MTES>50% glycerol+50% MTES>no MTES. Although MTES addition improved observational hardness, MTES also promoted sol-gel surface cracks, which potentially exacerbates BC leakage issues. Increased cracking occurred from recipes in the following rank: 10% glycerol+20% MTES>20% glycerol+20% MTES or 10% glycerol+50% MTES>50% glycerol+50% MTES>20% glycerol+50% MTES>no MTES. To summarize, addition of 20% glycerol+50% MTES lessened cracking and improved observational hardness for the final gel plate coating.

Cracking during the fast-drying process when mechanical stress exceeded the material ability to elastically respond has been reported; (40) it was found that adding MTES led to higher hardness and caused less elastic gel and cracking. To mitigate the cracking issue from MTES, a slower drying process and high moisture environment during gelation can be helpful. A plate dish cover was therefore used to slow sol-gel evaporation and control aging time before transferring to solution. The optimal aging time under dish cover was 19 hours (TEOS only recipe) and 15 mins (MTES addition recipe). Both procedures completely encapsulated BCs and resulting gel plates exhibited high observational hardness with no observed biochar escape after one week of shaking.

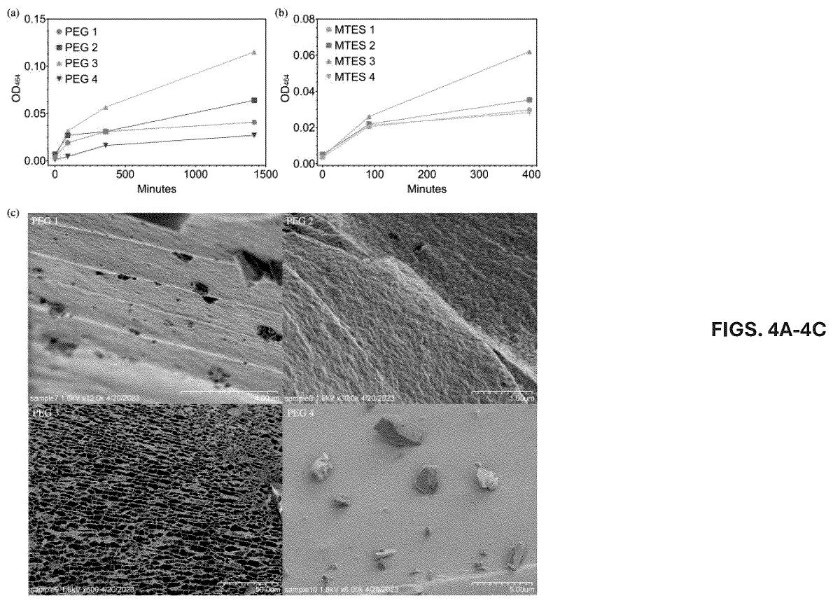

Impacts on pore structure: Although the gel plate coated BCs, produced by controlling aging time for both only TEOS and MTES addition recipes, could achieve complete BC surface coating and prevent BC escape after continuous shaking for one week, this improvement on encapsulation hampered benzoate biodegradation when testing encapsulated biofilm-enriched BCs. In contrast, incomplete encapsulation of biofilm-BCs, that could allow biofilm-BCs to escape the gel plate, did display benzoate biodegradation. It was hypothesized that the no benzoate degradation phenomenon observed with encapsulated gel plates was due to gel pore size limiting pollutant mass transfer from the surrounding solution into coated biofilms. Glycerol and PEG addition are reported to increase pore size and improve biocompatibility. (20) The diffusion capability was tested of chemicals through existing pore size and the impacts that adding glycerol (0%, 6%, and 20%, v/v of sol solution) and PEG400 (0%, 10% and 20%, v/v of sol solution) in sol-gel recipe had on manipulating pore size. Methyl orange was added in the sol solution prior to gelation on the premise that if the pore size of gel increased, methyl orange would more easily diffuse and be detected at OD464. Based on this premise, the gel pore size was larger when either glycerol or PEG400 was added compared to no glycerol or PEG400 addition (FIGS. 4A and 4B). More specifically, a higher volume of PEG400 addition resulted in larger gel pore size, while glycerol did not increase the pore size of the gel recipe with MTES addition, but did help with the only TEOS recipe. Similarly, the gel surface was rougher and more porous when more PEG400 was added while no PEG400 or glycerol addition group resulted in a smooth surface with no pores (FIG. 4C). Overall, the gel pore size with the tested glycerol and PEG400 addition ranked follows: 20% PEG400>10% PEG400>6% glycerol>no glycerol or PEG400.

Cell Viability Enhancement: Individually Encapsulated Biofilm-BC Protects Cell Viability for at Least 3 Months without Carbon Source Supply.

The viability of biofilms on BC encapsulated with two different recipes were compared before and after treatment with non-encapsulated biofilms by visualizing live and dead biomass on the BC surfaces. The two recipes evaluated were the optimal tea ball coating (recipe-E&F in FIG. 3), which formed the least cracks and was the most smooth, even, and complete sol-gel surface; and phosphate buffer addition (recipe-A in FIG. 3), which maintained cell viability after coating as evidenced by acetate and benzoate biodegradation (chemical degradation data are presented below).

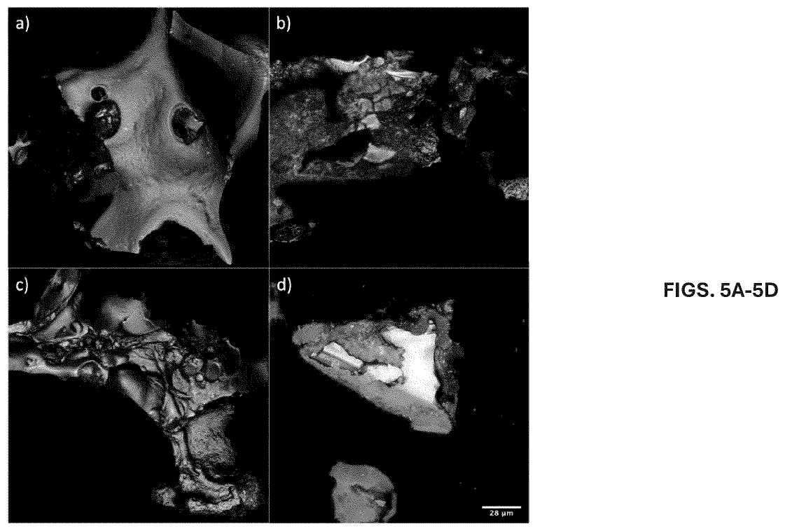

Before treatment, live LB400 cells (green dots) were observed underneath the sol-gel made by recipe-A while partial BC surface was covered by sol-gel (continuous green fluorescence, FIG. 5B). Following treatment, both non-coated biofilms and biofilms coated by sol-gel recipe-A detached from BC surfaces with only limited amounts of LB400 cells remaining within the BC pore structure (FIGS. 5A and 5C). Sol-gel made by recipe-A also disappeared after treatment. This indicates that biofilm abundance, without sol-gel protection, decreased over time against continuous shear force, and sol-gel made by recipe-A only maintains gel integrity and protects biomass for less than 25 days. In contrast, after three months, sol-gel created by recipe-E&F still covered most BC surfaces as shown by continuous green fluorescence (FIG. 5D). Live cells (green dots) were noted inside the gel, although dead cells (red dots) were seen nearby the gel. This finding demonstrates that sol-gel made with recipe-E&F not only prevented biomass from being washed away by continuous application of shear force, but also protected cell viability in the absence of carbon and energy sources for at least three months. In general, sol-gel made with recipe-E&F outperformed that with recipe-A in terms of complete biofilm-BC coating, maintaining sol-gel, and more importantly, biofilm integrity.

### Chemical Degradation Proof-of-Concept: Individually Coated Biofilm-BC Exhibited Enhanced Biodegradation Potential Over Gel Plate-Coated Biofilm.

To evaluate the biodegradation potential of coated biofilms, benzoate and acetate were chosen to compare carbon source degradation performance between sol-gel coated biofilms and non-coated biofilms. Acetate degradation is the first indication of the activity of coated biofilms, while benzoate is an inducer and product of PCB biodegradation pathway (bph) and is more easily measured in the aqueous phase than PCBs. (41) Three sol-gel coating recipes were chosen:

phosphate buffer addition (recipe-A), gel plate that completely encapsulates biochar without escape (gel plate recipe), and individual coating by tea ball (adjusted recipe-E and recipe-F with single tea ball coating in FIG. 3). Both non-coated biofilms and biofilms coated by recipe-A degraded benzoate well during three rounds of biofilm reuse (FIG. 6A). Compared with non-coated biofilms, coated biofilms lagged for two days prior to consuming benzoate and acetate (FIG. 6A (benzoate)). Recipe-A coated biofilms degraded about 30% more benzoate than non-coated biofilms (p=0.072, significant differences of remaining benzoate concentration for two groups at day-12) in the beginning of the second round of biofilm reuse, but the benzoate degradation differences were absent in the third round of biofilm recycle. This phenomenon may be related to insufficient biofilm protection by recipe-A sol-gel (FIGS. 6A and 6C), where biofilms and sol-gel detached from the BC surface within 25 days of treatment even with sol-gel encapsulation (FIG. 5). Thus, for recipe-A, most of the biofilms, both non-coated and coated, were resuspended into the aqueous solution, and only partial biofilms were left on BC surface and brought over to fresh media in each round of reuse, resulting in the same degradation performance.

When different cell-to-sol solution ratios were applied to coat biofilms, both benzoate and acetate were readily degraded by coated biofilms and cells within three days, respectively. A higher ratio (4:1) degraded acetate significantly (p<0.0019) faster than that of 2:1 and 3:1 (4:1:0.048 mM/hr, 2:1 and 3:1:0.0012 mM/hr) in the first half degradation period, but cell leaching was also observed together with carbon source biodegradation.

Although the sol-gel pore size was controlled to allow chemical transfer for gel plate coated biofilms (FIG. 6B), there was still no benzoate degradation by both TEOS only gel and MTES addition gel after 9 and 13 days. One hypothesis for this phenomenon is that the high degree of sol-gel mass transfer resistance delayed benzoate diffusion to biofilms from the bulk liquid. The square-shaped gel plate did indeed prevent biomass from escaping; however, the encapsulation made biofilms contact with the chemicals difficult. In contrast, biofilms individually coated by adjusted recipe-E&F could completely degrade benzoate within 12 days (FIG. 6C), where individual coating maintained the original granular structure of biofilm-BC and did not limit the direct contact between biofilms and chemicals. However, glycerol addition degraded benzoate marginally more efficiently (10 days) than PEG400 addition (12 days), potentially because of sol-gel porosity and pore size. Overall, although biofilms coated by recipe-A and adjusted recipe-E&F both degraded benzoate efficiently, recipe-A had severe biofilm escape issues and unstable gel integrity that may facilitate benzoate degradation, whereas the gel plate coated biofilms encountered degradation difficulty due to potential mass transfer limitations. To balance the trade-off between biodegradation efficiency and biofilm protection, adjusted recipe-E&F were optimal, but recipes can be tuned to match other aims/compounds as well.

The results demonstrate that individual encapsulation in a matrix from TEOS and MTES precursors and incorporating glycerol/PEG400 and PVP are effective in preserving biofilm viability and activity and transferring chemicals from the bulk liquid to the biofilm microenvironment on the BC surface. This work provides a platform for developing biofilm protection applications with different microbial biofilm types (i.e., aerobic and potentially anaerobic) growing on or attached to different solid surfaces. Moreover, given the high porosity and pore size of sol-gel, target pollutants with radius sizes varying from <75 nm to <27.5 nm can pass through the sol-gel depending on glycerol or PEG400 addition. These size ranges encompass a large array of chemicals, where PCBs have molecular size of at most ~1.3 nm. The porous structure of sol-gel matrix also allows for application in bioreactors where fluid with pollutants can pass over coated biofilms, and encapsulated biofilms could be reused given its high stability of encapsulant and maintained biomass and activity.

This demonstrates the capacity of TEOS+MTES tuned sol-gel matrix for protecting biofilms that can be applied to in situ contaminated sediment remediation. The encapsulated biofilms can sustain and even prolong their biodegradation potential against adverse environmental conditions, such as limited carbon source supply and undesirable changes including high salinity and continuous shear force. A sol-gel matrix not only preserves high viable cell density given its high observational hardness and stability, but also prevents biofilm maturation and detachment because the sol-gel restrains cell outgrowth. (20) Overall, this novel development of sol-gel application on biofilms can enhance longevity of cell activity and further extend the biodegradation time.

### Encapsulated Biofilms Degraded Three Different PCBs (PCB4, PCB18 and PCB52) Over a 45-Day Period as Compared to Unencapsulated Biofilms.

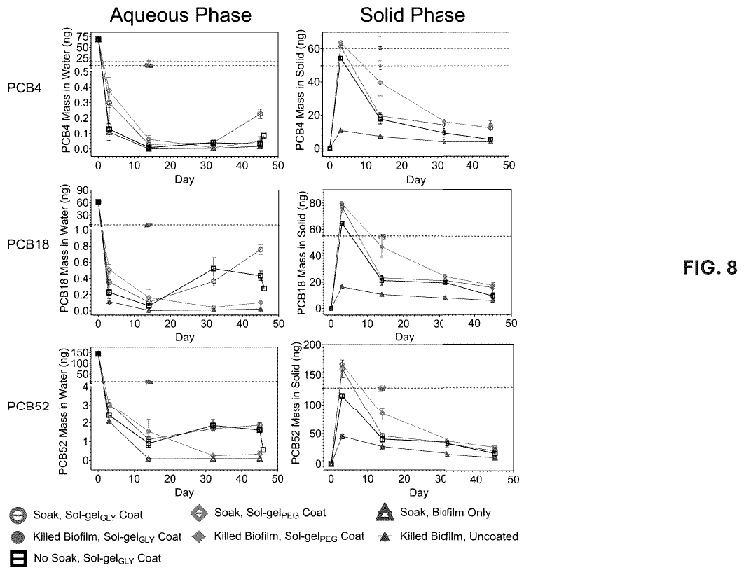

FIG. 8 demonstrates changes in PCB congener mass over time in the aqueous (column 1) and solid (column 2) phases, in the presence of uncoated and sol-gel coated LB400 biofilms on CKB: PCB4 (row 1), PCB 18 (row 2), and PCB52 (row 3). PCBs in both aqueous and solid phases were measured at each time point, where solid phase PCBs were considered sorbed to materials (sol-gel and/or CKB). The differences between abiotic controls and biotic treatments were considered PCB biodegradation by biofilms.

BphA Expression Levels (i.e., Transcript/Gene Ratios) were Elevated in the Encapsulated Biofilms Compared to the Unencapsulated Biofilms.

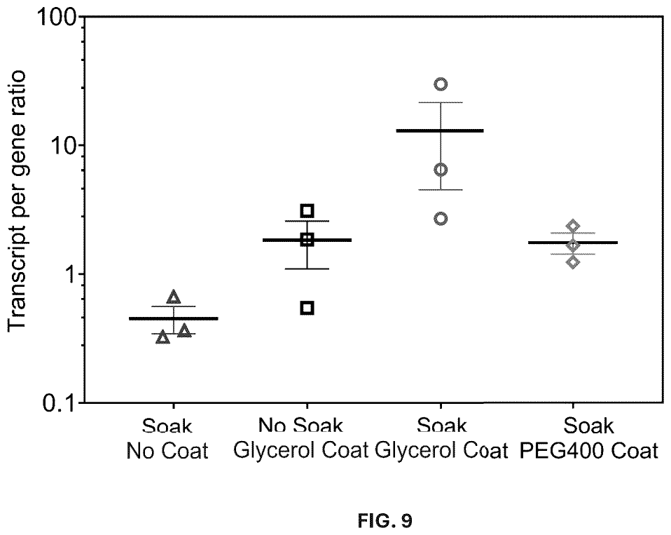

FIG. 9 demonstrates BphA expression levels (transcript per gene ratios=transcript abundance/gene abundance) of sol-gel coated and uncoated biofilms under standard conditions at day-45.

### Conclusions

Herein the disclosure of the first to develop novel method for encapsulating PCB-degrading aerobic biofilm-enriched biochar with sol-gel. Prior studies have focused more on encapsulation of living cells in sol-gel matrix mainly for storage purposes, (21, 25, 29) but no study has successfully immobilized biofilms growing on solid surface for remediation. The surprising features of this sol-gel approach lie in its capability to protect coated biofilms against adverse environmental changes (i.e., shear force and high salinity) and maintain biofilm viability and activity via its porous, well-covered, and mechanical stable gel matrix. Particularly, these environments are normally unfavorable to microbial cells (e.g., suspended cells and non-coated biofilms). (19,44) The method allows coated biofilms to remain viable without carbon sources for over three months, which exhibits great potential to extend biodegradation reaction time.

This also provide methods for improving in situ sediment remediation approaches that mitigate exposure to a pressing environmental toxicant. LC-PCBs, as the most volatile PCBs, cause direct exposure from sediment to humans and ecosystems. This newly developed approach can extend LC-PCB biodegradation activity by protective sol-gel layer on biofilm-enriched biochar, and sol-gel encapsulated biofilms offer the advantages of high stability and long-time survival in harsh environments. This novel approach can therefore improve bioremediation efforts that decrease the PCB mass load, and other contaminates, in sediment and mitigate the sediment to air exposure pathway, thereby benefiting public health and ecosystems.

### BIBLIOGRAPHY

1. Saktrakulkla, P.; Lan, T.; Hua, J.; Marek, R. F.; Thorne, P. S.; Hornbuckle, K. C. Polychlorinated Biphenyls in Food. Environ. Sci. Technol. 2020, 54 (18), 11443-11452.
2. Bhavsar, S. P.; Jackson, D. A.; Hayton, A.; Reiner, E. J.; Chen, T.; Bodnar, J. Are PCB levels in fish from the Canadian Great Lakes still declining? J. Gt. Lakes Res. 2007, 33 (3), 592-605.
3. Allen-Gil, S.; Gubala, C.; Wilson, R.; Landers, D.; Wade, T.; Sericano, J.; Curtis, L. Organochlorine pesticides and polychlorinated biphenyls (PCBs) in sediments and biota from four US Arctic lakes. Arch. Environ. Contam. Toxicol. 1997, 33 (4), 378-387.
4. Nelson, E. D.; McConnell, L. L.; Baker, J. E. Diffusive exchange of gaseous polycyclic aromatic hydrocarbons and polychlorinated biphenyls across the air-water interface of the Chesapeake
   Bay. Environ. Sci. Technol. 1998, 32 (7), 912-919.
5. Heiger-Bernays, W. J.; Tomsho, K. S.; Basra, K.; Petropoulos, Z. E.; Crawford, K.; Martinez, A.; Hornbuckle, K. C.; Scammell, M. K. Human health risks due to airborne polychlorinated biphenyls are highest in New Bedford Harbor communities living closest to the harbor. Sci. Total Environ. 2020, 710, 135576.
6. Martinez, A.; Hadnott, B. N.; Awad, A. M.; Herkert, N. J.; Tomsho, K.; Basra, K.; Scammell, M. K.; Heiger-Bernays, W.; Hornbuckle, K. C. Release of Airborne Polychlorinated Biphenyls from New Bedford Harbor Results in Elevated Concentrations in the Surrounding Air. Environ. Sci. Technol. Lett. 2017, 4 (4), 127-131.
7. Martinez, A.; Wang, K.; Hornbuckle, K. C. Fate of PCB Congeners in an Industrial Harbor of Lake Michigan. Environ. Sci. Technol. 2010, 44 (8), 2803-2808.
8. Wang, H.; Adamcakova-Dodd, A.; Lehmler, H.-J.; Hornbuckle, K. C.; Thorne, P. S. Toxicity assessment of 91-day repeated inhalation exposure to an indoor school air mixture of PCBs. Environ. Sci. Technol. 2022, 56 (3), 1780-1790.
9. Li, X.; Hefti, M. M.; Marek, R. F.; Hornbuckle, K. C.; Wang, K.; Lehmler, H.-J. Assessment of polychlorinated biphenyls and their hydroxylated metabolites in postmortem human brain samples: age and brain region differences. Environ. Sci. Technol. 2022, 56 (13), 9515-9526.
10. Seegal, R. F. Epidemiological and Laboratory Evidence of PCB-Induced Neurotoxicity. Crit. Rev. Toxicol. 1996, 26 (6), 709-737.
11. Rice, C. P.; White, D. S. PCB availability assessment of river dredging using caged clams and fish. Environ. Toxicol. Chem. Int. J. 1987, 6 (4), 259-274.
12. Voie, Ø. A.; Johnsen, A.; Rossland, H. K. Why biota still accumulate high levels of PCB after removal of PCB contaminated sediments in a Norwegian fjord. Chemosphere 2002, 46 (9-10), 1367-1372.
13. Michaels, R. A.; Oko, U. M. Excessive PCBs in the Hudson River: attributable to incompleteness of dredging, or to seven years of dredging? Environ. Claims J. 2017, 29 (2), 115-140.
14. Payne, R. B.; Fagervold, S. K.; May, H. D.; Sowers, K. R. Remediation of polychlorinated biphenyl impacted sediment by concurrent bioaugmentation with anaerobic halorespiring and aerobic degrading bacteria. Environ. Sci. Technol. 2013, 47 (8), 3807-3815.
15. Bako, C. M.; Martinez, A.; Ewald, J. M.; Hua, J. B. X.; Ramotowski, D. J.; Dong, Q.; Schnoor, J. L.; Mattes, T. E. Aerobic bioaugmentation to decrease polychlorinated biphenyl (PCB) emissions from contaminated sediments to air. Environ. Sci. Technol. 2022, 56 (20), 14338-14349.
16. Dong, Q.; LeFevre, G. H.; Mattes, T. E. Black Carbon Impacts on Paraburkholderia xenovorans Strain LB400 Cell Enrichment and Activity: Implications toward Lower-Chlorinated Polychlorinated Biphenyls Biodegradation Potential. Environ. Sci. Technol. 2024, 58 (8), 3895-3907.
17. Payne, R. B.; Ghosh, U.; May, H. D.; Marshall, C. W.; Sowers, K. R. Mesocosm studies on the efficacy of bioamended activated carbon for treating PCB-impacted sediment. Environ. Sci. Technol. 2017, 51 (18), 10691-10699.
18. Payne, R. B.; Ghosh, U.; May, H. D.; Marshall, C. W.; Sowers, K. R. A pilot-scale field study: in situ treatment of PCB-impacted sediments with bioamended activated carbon. Environ. Sci. Technol. 2019, 53 (5), 2626-2634.
19. Egorova, D.; Pervova, M.; Demakov, V.; Plotnikova, E. Specific features of chlorinated biphenyl decomposition by Rhodococcus wratislaviensis strain KT112-7 under high salt conditions. Appl. Biochem. Microbiol. 2018, 54 (3), 252-261.
20. Dickson, D. J.; Page, C. J.; Ely, R. L. Photobiological hydrogen production from Synechocystis sp. PCC 6803 encapsulated in silica sol-gel. Int. J. Hydrog. Energy INT J HYDROGEN ENERG 2009, 34 (1), 204-215.
21. Desimone, M. F.; De Marzi, M. C.; Copello, G. J.; Fernández, M. M.; Malchiodi, E. L.; Diaz, L. E. Efficient preservation in a silicon oxide matrix of Escherichia coli, producer of recombinant proteins. Appl. Microbiol. Biotechnol. 2005, 68 (6), 747-752.
22. Tuttolomondo, M. V.; Alvarez, G. S.; Desimone, M. F.; Diaz, L. E. Removal of azo dyes from water by sol-gel immobilized Pseudomonas sp. J. Environ. Chem. Eng. 2014, 2 (1), 131-136.
23. Kandimalla, V. B.; Tripathi, V. S.; Ju, H. Immobilization of biomolecules in sol-gels: biological and analytical applications. Crit Rev Anal Chem 2006, 36 (2), 73-106.
24. Bhatia, R. B.; Brinker, C. J.; Gupta, A. K.; Singh, A. K. Aqueous sol-gel process for protein encapsulation. Chem. Mater. 2000, 12 (8), 2434-2441.
25. Nassif, N.; Bouvet, O.; Rager, M. N.; Roux, C.; Coradin, T.; Livage, J. Living bacteria in silica gels. Nat. Mater. 2002, 1 (1), 42-44.
26. Mukerjee-Dhar, G.; Shimura, M.; Kimbara, K. Degradation of polychlorinated biphenyl by cells of Rhodococcus opacus strain TSP203 immobilized in alginate and in solution. Enzyme Microb. Technol. 1998, 23 (1-2), 34-41.
27. Mutlu, B. R.; Yeom, S.; Tong, H.-W.; Wackett, L. P.; Aksan, A. Silicon alkoxide cross-linked silica nanoparticle gels for encapsulation of bacterial biocatalysts. J. Mater. Chem. A 2013, 1 (36), 11051-11060.
28. Mehner, A.; Dong, J.; Prenzel, T.; Datchary, W.; Lucca, D. A. Mechanical and chemical properties of thick hybrid sol-gel silica coatings from acid and base catalyzed sols. J Solgel Sci Technol 2010, 54 (3), 355-362.
29. Alvarez, G. S.; Desimone, M. F.; Diaz, L. E. Immobilization of bacteria in silica matrices using citric acid in the sol-gel process. Appl. Microbiol. Biotechnol. 2007, 73 (5), 1059-1064.
30. Dudás, Z.; Len, A.; Ianăşi, C.; Paladini, G. Structural modifications caused by the increasing MTES amount in hybrid MTES/TEOS-based silica xerogels. Mater. Charact. 2020, 167, 110519.
31. Desimone, M. F.; Alvarez, G. S.; Foglia, M. L.; Diaz, L. E. Development of sol-gel hybrid materials for whole cell immobilization. Recent Pat Biotechnol. 2009, 3 (1), 55-60.
32. Dickson, D. J.; Ely, R. L. Evaluation of encapsulation stress and the effect of additives on viability and photosynthetic activity of Synechocystis sp. PCC 6803 encapsulated in silica gel. Appl. Microbiol. Biotechnol. 2011, 91 (6), 1633-1646.
33. Conroy, J. F.; Power, M. E.; Martin, J.; Earp, B.; Hosticka, B.; Daitch, C. E.; Norris, P. M. Cells in sol-gels I: a cytocompatible route for the production of macroporous silica gels. J Solgel Sci Technol 2000, 18 (3), 269-283.
34. Jiang, J.; Shen, Y.; Yu, D.; Yang, T.; Wu, M.; Yang, L.; Petru, M. Porous Film Coating Enabled by Polyvinyl Pyrrolidone (PVP) for Enhanced Air Permeability of Fabrics: The Effect of PVP Molecule Weight and Dosage. Polymers (Basel) 2020, 12 (12), 2961.
35. Kurakula, M.; Rao, G. Pharmaceutical assessment of polyvinylpyrrolidone (PVP): As excipient from conventional to controlled delivery systems with a spotlight on COVID-19 inhibition. J Drug Deliv Sci Technol 2020, 60, 102046.
36. Mehner, A.; Dong, J.; Prenzel, T.; Datchary, W.; Lucca, D. Mechanical and chemical properties of thick hybrid sol-gel silica coatings from acid and base catalyzed sols. J Solgel Sci Technol 2010, 54 (3), 355-362.
37. Sharma, T. Biochar and other properties resulting from the gasification and combustion of biomass with different components. The University of Iowa, 2019.
38. Brinker, C. J. Hydrolysis and condensation of silicates: effects on structure. Journal of non-crystalline solids 1988, 100 (1-3), 31-50.
39. Wu, L.; Liu, X.; Lv, G.; Zhu, R.; Tian, L.; Liu, M.; Li, Y.; Rao, W.; Liu, T.; Liao, L. Study on the adsorption properties of methyl orange by natural one-dimensional nano-mineral materials with different structures. Sci. Rep. 2021, 11 (1), 10640.
40. Kappert, E. J.; Pavlenko, D.; Malzbender, J.; Nijmeijer, A.; Benes, N. E.; Tsai, P. A. Formation and prevention of fractures in sol-gel-derived thin films. Soft matter 2015, 11 (5), 882-888.
41. Denef, V. J.; Patrauchan, M. A.; Florizone, C.; Park, J.; Tsoi, T. V.; Verstraete, W.; Tiedje, J. M.; Eltis, L. D. Growth substrate- and phase-specific expression of biphenyl, benzoate, and C1 metabolic pathways in Burkholderia xenovorans LB400. J Bacteriol 2005, 187 (23), 7996-8005.
42. Zhang, X.; Wang, H.; Li, Q.; Yin, Z.; Qi, H.; Yang, J.; Wang, X.; Xiao, W.; Zhang, L. Development of Organogels for Live Yarrowia lipolytica Encapsulation. J. Am. Chem. Soc. 2022, 144 (23), 10251-10258.
43. Hoyos-Leyva, J. D.; Bello-Perez, L. A.; Agama-Acevedo, E.; Alvarez-Ramirez, J. Thermodynamic analysis for assessing the physical stability of core materials microencapsulated in taro starch spherical aggregates. Carbohydrate Polymers 2018, 197, 431-441.
44. Ding, P.; Wu, P.; Jie, Z.; Cui, M.-H.; Liu, H. Damage of anodic biofilms by high salinity deteriorates PAHs degradation in single-chamber microbial electrolysis cell reactor. Sci. Total Environ. 2021, 777, 145752.

### STATEMENTS OF THE INVENTION

1. A microbial composition comprising bacteria and a feedstock, wherein the microbial composition is coated with a sol gel composition.
2. The microbial composition of statement 1, further comprising one or more biofilms produced by the bacteria.
3. The microbial composition of statement 1 or 2, wherein the bacteria are selected from the genera of Bacillus, Rhodococcus, Arthrobacter, Cycloclasticus, Paraburkholderia, Alcanivorex or combinations thereof.
4. The microbial composition of any one of statements 1 to 3, wherein the bacteria are Paraburkholderia.
5. The microbial composition of claim statement, wherein the bacteria are Paraburkholderia xenovorans (e.g., Paraburkholderia xenovorans LB400).
6. The microbial composition of any one of statements 1 to 3, wherein the bacteria Alcanivorax borkumensis I.
7. The microbial composition of any one of statements 1 to 6, wherein the feedstock is black carbon.
8. The microbial composition of statement 7, wherein the black carbon is biochar.
9. The microbial composition of any one of statements 1 to 8, wherein the feedstock is corn kernel biochar.
10. The microbial composition of any one of statements 1 to 9, wherein the sol gel comprises tetraethyl orthosilicate (TEOS).
11. The microbial composition of any one of statements 1 to 10, wherein the sol gel comprises methyltriethoxysilane (MTES).
12. The microbial composition of any one of statements 1 to 11, wherein the sol gel comprises glycerol.
13. The microbial composition of any one of statements 1 to 12, wherein the sol gel comprises polyvinylpyrrolidone (PVP).
14. The microbial composition of any one of statements 1 to 13, wherein the sol gel comprises polyethylene glycol (PEG).
15. The microbial composition of statement 14, wherein the PEG is PEG400.
16. The microbial composition of any one of statements 1 to 15, wherein the sol gel is formed from MTES/TEOS/H₂O in a ratio of 0.5:0.5:20, 10 mg PVP and 20% glycerol (v/v).
17. The microbial composition of any one of statements 1 to 11 or 13 to 15, wherein the sol gel is formed MTES/TEOS/H₂O in a ratio of 0.5:0.5:20, 10 mg PVP, and 20% PEG400 (v/v).
18. The microbial composition of any one of statements 1 to 17, wherein the sol gel has pores larger than a contaminant molecule and smaller than the bacteria.
19. The microbial composition of any one of statements 1 to 18, microbial composition is coated with two coats of the sol gel composition.
20. The microbial composition of any one of statements 2 to 19, wherein stability of the biofilm is increased as compared to a microbial composition that is not coated with a sol gel composition.
21. The microbial composition of any one of statements 1 to 5 or 7 to 17, wherein the bacteria degrade polychlorinated biphenyl (PCB).
22. The microbial composition of statement 21, wherein the PCB is PCB4, PCB18, PCB52 or combination thereof.
23. A method for bioremediation comprising contacting a contaminated environmental sample with the microbial composition of any one of statements 1 to 22.
24. The method of statement 23, wherein the is selected from among soil, water, and air samples.
25. The method of statement 23, wherein the sample is in situ.
26. A method for increasing the stability of a microbial composition comprising bacteria and a feedstock, the method comprising coating the microbial composition with a sol gel composition.
27. The method of statement 26, further comprising one or more biofilms produced by the bacteria.
28. The method of statement 26 or 27, wherein the bacteria are selected from the genera of Bacillus, Rhodococcus, Arthrobacter, Cycloclasticus, Paraburkholderia, Alcanivorex or combinations thereof.
29. The method of any one of statements 26 to 28, wherein the bacteria are Paraburkholderia.
30. The method of statement 29, wherein the bacteria are Paraburkholderia xenovorans (e.g., Paraburkholderia xenovorans LB400).
31. The method of any one of statements 26 to 27, wherein the bacteria are Alcanivorax borkumensis I.
32. The method of any one of statements 26 to 31, wherein the feedstock is black carbon.
33. The method of statement 32, wherein the black carbon is biochar.
34. The method of any one of statements 26 to 31, wherein the feedstock is corn kernel biochar.
35. The method of any one of statements 26 to 34, wherein the sol gel comprises tetraethyl orthosilicate (TEOS).
36. The method of any one of statements 26 to 34, wherein the sol gel comprises methyltriethoxysilane (MTES).
37. The method of any one of statements 26 to 36, wherein the sol gel comprises glycerol.
38. The method of any one of statements 26 to 37, wherein the sol gel comprises polyvinylpyrrolidone (PVP).
39. The method of any one of statements 26 to 38, wherein the sol gel comprises polyethylene glycol (PEG).
40. The method of statement 39, wherein the PEG is PEG400.
41. The method of any one of statements 26 to 40, wherein the sol gel is formed from MTES/TEOS/H₂O in a ratio of 0.5:0.5:20, 10 mg PVP and 20% glycerol (v/v).
42. The method of any one of statements 26 to 40, wherein the sol gel is formed MTES/TEOS/H₂O in a ratio of 0.5:0.5:20, 10 mg PVP, and 20% PEG400 (v/v).
43. The method of any one of statements 26 to 42, wherein the sol gel has pores larger than a contaminant molecule and smaller than the bacteria.
44. The method of any one of statements 26 to 43, microbial composition is coated with two coats of the sol gel composition.
45. The method of any one of statements 26 to 44, wherein the bacteria degrade polychlorinated biphenyl (PCB).
46. The method of any one of statements 26 to 45, wherein expression levels of bphA is increased as compared to unencapsulated biofilms.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A microbial composition comprising bacteria and a feedstock, wherein the microbial composition is coated with a sol gel composition.

2. The microbial composition of claim 1, further comprising one or more biofilms produced by the bacteria.

3. The microbial composition of claim 1, wherein the bacteria are selected from the genera of Bacillus, Rhodococcus, Arthrobacter, Cycloclasticus, Paraburkholderia, Alcanivorex or combinations thereof.

4. The microbial composition of claim 1, wherein the feedstock is black carbon.

5. The microbial composition of claim 4, wherein the black carbon is biochar.

6. The microbial composition of claim 1, wherein the sol gel comprises tetraethyl orthosilicate (TEOS).

7. The microbial composition of claim 1, wherein the sol gel comprises methyltriethoxysilane (MTES).

8. The microbial composition of claim 1, wherein the sol gel is formed from MTES/TEOS/H₂O in a ratio of 0.5:0.5:20, 10 mg PVP and 20% glycerol (v/v).

9. The microbial composition of claim 1, wherein the sol gel is formed MTES/TEOS/H₂O in a ratio of 0.5:0.5:20, 10 mg PVP, and 20% PEG400 (v/v).

10. The microbial composition of claim 1, wherein the sol gel has pores larger than a contaminant molecule and smaller than the bacteria.

11. The microbial composition of claim 1, wherein the microbial composition is coated with two coats of the sol gel composition.

12. The microbial composition of claim 2, wherein stability of the biofilm is increased as compared to a microbial composition that is not coated with a sol gel composition.

13. A method for bioremediation comprising contacting a contaminated environmental sample with the microbial composition of claim 1.

14. The method of claim 13, wherein the is selected from among soil, water, and air samples.

15. A method for increasing the stability of a microbial composition comprising bacteria and a feedstock, the method comprising coating the microbial composition with a sol gel composition.

16. The method of claim 15, further comprising one or more biofilms produced by the bacteria.

17. The method of claim 15, wherein the bacteria are selected from the genera of Bacillus, Rhodococcus, Arthrobacter, Cycloclasticus, Paraburkholderia, Alcanivorex or combinations thereof.

18. The method of claim 15, wherein the feedstock is black carbon.

19. The method of claim 15, wherein the black carbon is biochar.

20. The method of claim 15, wherein the sol gel comprises tetraethyl orthosilicate (TEOS).

21. The method of claim 15, wherein the sol gel comprises methyltriethoxysilane (MTES).

22. The method of claim 15, wherein the sol gel is formed from MTES/TEOS/H₂O in a ratio of 0.5:0.5:20, 10 mg PVP and 20% glycerol (v/v).

23. The method of claim 15, wherein the sol gel is formed MTES/TEOS/H₂O in a ratio of 0.5:0.5:20, 10 mg PVP, and 20% PEG400 (v/v).

24. The method of claim 15, wherein the sol gel has pores larger than a contaminant molecule and smaller than the bacteria.

25. The method of claim 15, wherein the microbial composition is coated with two coats of the sol gel composition.

26. The method of claim 15, wherein the bacteria degrade polychlorinated biphenyl (PCB).

27. The method of claim 15, wherein expression levels of bphA is increased as compared to unencapsulated biofilms.
